(19) United States
(12) Patent Application Publication
(10) Pub. No.: US 2010/0076311 A1
(43) Pub. Date: Mar. 25, 2010

(54) THICK-SLICE PROCESSING AND DISPLAY OF INFORMATION FROM A VOLUMETRIC ULTRASOUND SCAN OF A CHESTWARDLY COMPRESSED BREAST

(75) Inventors: Laszlo Tabar, Cave Creek, AZ (US); Shih-Ping Wang, Los Altos, CA (US); Tor C. Anderson, Los Gatos, CA (US); Jiayu Chen, Palo Alto, CA (US)

Correspondence Address: COOPER & DUNHAM, LLP, 30 Rockefeller Plaza, 20th Floor, NEW YORK, NY, 10112, US

(73) Assignee: U-SYSTEMS, INC., San Jose, CA (US)

(21) Appl. No.: 12/441,975

(22) PCT Filed: Sep. 21, 2007
(86) PCT No.: PCT/US07/20538

§ 371 Date: Nov. 24, 2009

Related U.S. Application Data
(60) Provisional application No. 60/826,545, filed on Sep. 22, 2006.
(60) Provisional application No. 60/827,052, filed on Sep. 26, 2006.

(51) Int. Cl. A61B 8/14; G06K 9/00

(52) U.S. Cl. 600/443; 382/131

(57) ABSTRACT

Systems, methods, and related computer program products for acquiring, processing, and displaying breast ultrasound information are described. In one preferred embodiment, a three-dimensional data volume of a sonographic property of a chestwardly compressed breast is acquired, and the data volume is processed to generate a plurality of two-dimensional coronal thick-slice images. Each coronal thick-slice image is representative of the sonographic property within a thick-slice subvolume of the breast substantially parallel to a coronal plane. The thick-slice subvolume has a thickness selected for optimal viewing of terminal ductal lobular unit (TDLU) patterns in a display of the thick-slice images.

### CROSS-REFERENCE TO RELATED APPLICATIONS

This application claims the benefit of U.S. Provisional Application No. 60/826,545, filed Sep. 22, 2006, and U.S. Provisional Application No. 60/827,052, filed Sep. 26, 2006, each of which is incorporated by reference herein. The subject matter of this provisional patent specification is related to the subject matter of U.S. Ser. No. 10/997,293 (Pub. No. US 2005/0171430A1), which is a continuation-in-part of U.S. Ser. No. 10/305,936 (Pub. No. US 2003/0212327A1), which is a continuation-in-part of U.S. Ser. No. 10/160,836 (Pub. No. US 2003/0007598A1), which claims the benefit, inter alia, of U.S. Ser. No. 60/326,715, filed Oct. 3, 2001, each of which is incorporated by reference herein.

### FIELD

This patent specification relates to medical ultrasound imaging. More particularly, this patent specification relates to processing and/or display of breast ultrasound information for breast cancer screening and/or diagnosis purposes.

### BACKGROUND

Volumetric ultrasound scanning of the breast can serve as a complementary modality for breast cancer screening as described, for example, in the commonly assigned US 2003/0007598A1 and US 2003/0212327A1, each of which is incorporated by reference herein. Whereas a conventional two-dimensional x-ray mammogram only detects a summation of the x-ray opacity of individual slices of breast tissue over the entire breast, ultrasound can separately detect the sonographic properties of individual slices of breast tissue, and therefore may allow detection of breast lesions where x-ray mammography alone fails. Another well-known shortcoming of x-ray mammography practice is found in the case of dense-breasted women, including patients with high content of fibroglandular tissues in their breasts. Because fibroglandular tissues have higher x-ray absorption than the surrounding fatty tissues, portions of breasts with high fibroglandular tissue content are not well penetrated by x-rays and thus the resulting mammograms contain reduced information in areas where fibroglandular tissues reside.

Still another shortcoming of x-ray mammography practice relates to difficulty in imaging near the chest wall. A substantial number of cancers are known to occur within 3 cm of the chest wall. In the craniocaudal (CC) and mediolateral oblique (MLO) views common to x-ray mammography acquisition protocols, the breasts are flattened between compression plates extending outward from the chest wall, and the x-rays are directed through the compression plates in a direction roughly parallel to the chest wall. With regard to breast tissues near the chest wall, it is physically difficult to pull these tissues outward to locations between the compression plates, making it difficult to properly image these tissues and more likely that cancers in these tissues will be missed in the resultant x-ray mammograms.

In addition to being useful as a complementary modality to x-ray mammography, ultrasound mammography could well become a sole breast cancer screening modality for at least some patient groups. For example, it is believed that preventive health care policy will progress toward the adoption of regular breast cancer screening procedures for increasingly younger women, e.g., women under the age of 40, and perhaps even under the age of 30 if there is a family history of cancer. Because younger women generally have denser breasts, the shortcomings of conventional two-dimensional x-ray mammography are expected to become especially apparent. Even further, because the dangers of x-ray radiation exposure are cumulative over a lifetime, ultrasound mammography could well become a sole breast cancer screening modality for women in these younger age groups. Other demographics indicating higher breast densities among certain groups, regions, or countries may also lead to the increased adoption of breast ultrasound as a sole or adjunctive screening modality for those groups, regions, or countries.

Once a thorough set of breast ultrasound scans is obtained, a challenge arises in the context of processing and displaying the breast ultrasound information to a clinician. In general, there is an inherent tension between (i) promoting high sensitivity/specificity in the screening and/or diagnosis process, and (ii) promoting efficient patient throughput to keep costs manageable. Thus, for example, while careful slice-by-slice scrutiny of the raw ultrasound scans by a well-trained radiologist would promote high sensitivity and specificity, the overall workflow efficiency of this method would be low, and therefore costs would be high, in view of the hundreds of individual raw ultrasound slices to be reviewed for each patient.

Accordingly, it would be desirable to provide an interactive user interface for viewing breast ultrasound information that can be effective for (i) adjunctive ultrasound mammography environments in which the ultrasound information complements x-ray mammogram information, and/or (ii) ultrasound-only mammography environments in which ultrasound is a sole screening modality.

It would be further desirable to provide processing and display of breast ultrasound information in a manner that promotes high specificity and sensitivity in the breast cancer screening and/or diagnosis process.

### SUMMARY

A system, method, and computer program product for processing and displaying breast ultrasound information is provided, comprising receiving a three-dimensional data volume of a sonographic property of a chestwardly compressed breast and processing the data volume to generate a plurality of two-dimensional coronal thick-slice images, each coronal thick-slice image being representative of the sonographic property within a thick-slice subvolume of the breast substantially parallel to a, coronal plane, wherein the thick-slice subvolume has a thickness selected for optimal viewing of terminal ductal lobular unit (TDLU) patterns in a display of the thick-slice images. In one particularly useful preferred embodiment, the thick-slice subvolumes are roughly 2 mm in thickness, and the thick-slice images are displayed in cine fashion to a viewer in a succession corresponding to progressive depths toward and/or away from the chest wall. It has been found that the cine display of the thick-slice images provides striking facilitation of the perception of abnormal breast structures, such as those associated with cancer, when the thick-slice subvolumes are associated with the dimensions of breast TDLUs in terms of thickness.

According to another preferred embodiment, projection-view thick-slice images are displayed that correspond to slab-like subvolumes parallel to a projection-view plane, the projection-view plane being other than a coronal plane. Examples include CC-projection thick-slice images and MLO-projection thick-slice images, and are useful even though the breast volume was not flattened along those planes when the ultrasound volume was acquired. Preferably, the projection-view thick-slice images are displayed adjacent to corresponding x-ray mammogram views. According to another preferred embodiment, the projection-view thick-slice images are stretched, morphed, or otherwise remapped to have to have a lateral shape that more closely corresponds to the corresponding x-ray mammogram view, the resultant remapped projection-view thick-slice image thereby being more easily compared thereto to that x-ray mammogram.

### BRIEF DESCRIPTION OF THE DRAWINGS

FIG. 1 illustrates a conceptual diagram of a breast cancer screening and/or diagnosis system according to a preferred embodiment;

FIG. 2A illustrates terminal ductal lobular units (TDLUs) of a breast;

FIG. 2B illustrates a conceptual drawing of a TDLU;

FIG. 3A illustrates a conceptual example of a side view of a thick-slice subvolume of a breast;

FIG. 3B illustrates information illustrative of a staggered cine-like display of successive thick-slice subvolumes of a breast according to a preferred embodiment;

FIG. 4 illustrates a conceptual front view of a breast ultrasound volume acquired by ultrasonic scans of a chestwardly-compressed breast;

FIG. 5 illustrates a user display according to a preferred embodiment;

FIG. 6A illustrates remappable processing of a CC-projection thick-slice image (e.g., by stretching) to have a lateral shape that more closely corresponds to a corresponding CC-view x-ray mammogram according to a preferred embodiment;

FIG. 6B illustrates remappable processing of an MLO-projection thick-slice image (e.g., by stretching) to have a lateral shape that more closely corresponds to a corresponding MLO-view x-ray mammogram according to a preferred embodiment; and

FIG. 7 illustrates a user display according to a preferred embodiment.

### DETAILED DESCRIPTION

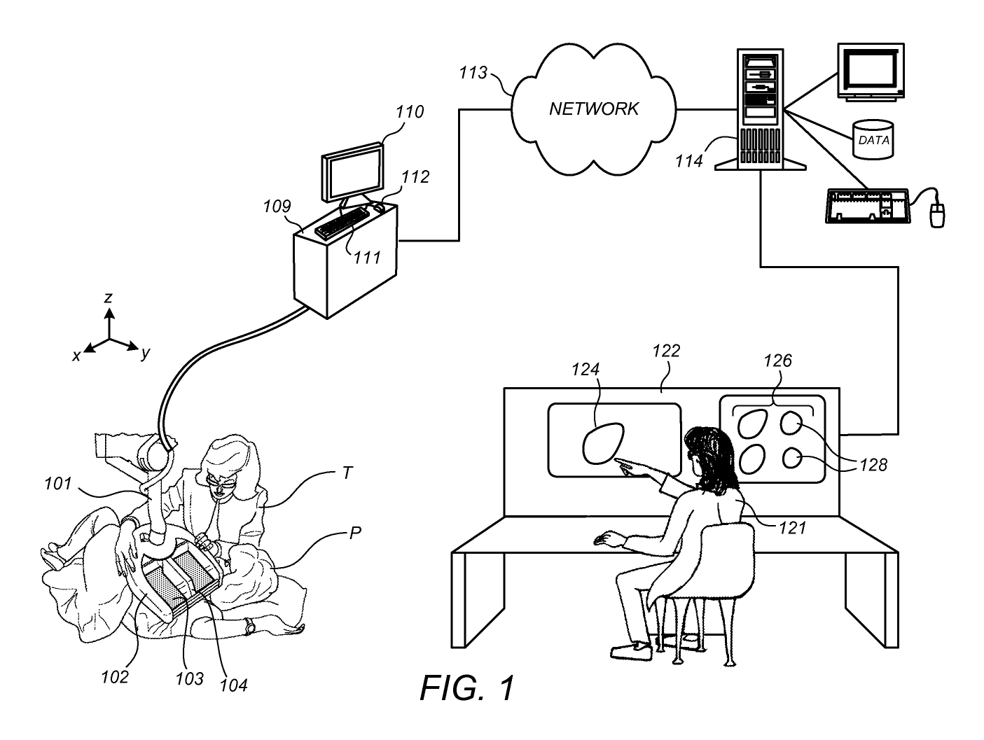

FIG. 1 illustrates a conceptual diagram of a breast cancer screening and/or diagnosis system according to a preferred embodiment. A medical professional such as an ultrasound technician T positions a scanning pod 102 located at the end of a mechanical arm 101 against the breast of a patient P, the scanning pod 102 comprising a substantially taut membrane 103 or other surface that compresses the breast in a generally chestward direction. An ultrasound probe 104 is automatically swept across a top surface of the membrane 103 to acquire scans of the breast therethrough. The probe 104 is actuated under control of an ultrasound processing unit 109 having a user interface including a display 110, keyboard 111, and mouse 112 or other graphical input device, which is usually controlled also by the technician T. In other preferred embodiments, the patient may be in a prone position or an upright position (not shown). By reducing the required ultrasonic penetration depth to the chest wall, scanning of a chestwardly compressed breast can occur at higher frequencies, e.g., 10-20 MHz, which can yield higher resolution images than for lower frequencies such as 5 MHz.

During or after the scanning process, the ultrasound scan data is provided across a computer network 113 to an ultrasound server 114 that processes and generates display information according to the functionalities described herein. The ultrasound server 114 may perform other HIS/RIS (hospital information system/radiology information system) activities such as archiving, scheduling, etc. It is to be appreciated that the processing of the ultrasound scan data may be performed by any of a variety of different computing devices coupled to the computer network 112 in various combinations without departing from the scope of the preferred embodiments. According to a preferred embodiment, a viewing workstation 122 is provided that provides a cine display 124 of a progression of coronal thick-slice images to a clinician 121, each coronal thick-slice image representing a sonographic property of the breast within a slab-like thick-slice subvolume thereof generally parallel to a coronal plane having a thickness that is optimal for viewing terminal ductal lobular unit (TDLU) structures in the breast, one particularly useful thickness being roughly 2 mm. Alternatively or in conjunction with the cine display 124, an array 126 of coronal thick-slice images 128 can be displayed for allowing simultaneous viewing of multiple coronal thick-slice images. In another preferred embodiment (not shown), the clinician is also provided with the ability to view individual planar ultrasound slices (along sagittal, axial, coronal, or other cut-planes through the three-dimensional breast volume), and the ability to view x-ray mammogram images or images from other modalities, as described further in US 2003/0007598A1 and/or US 2003/0212327A1, supra.

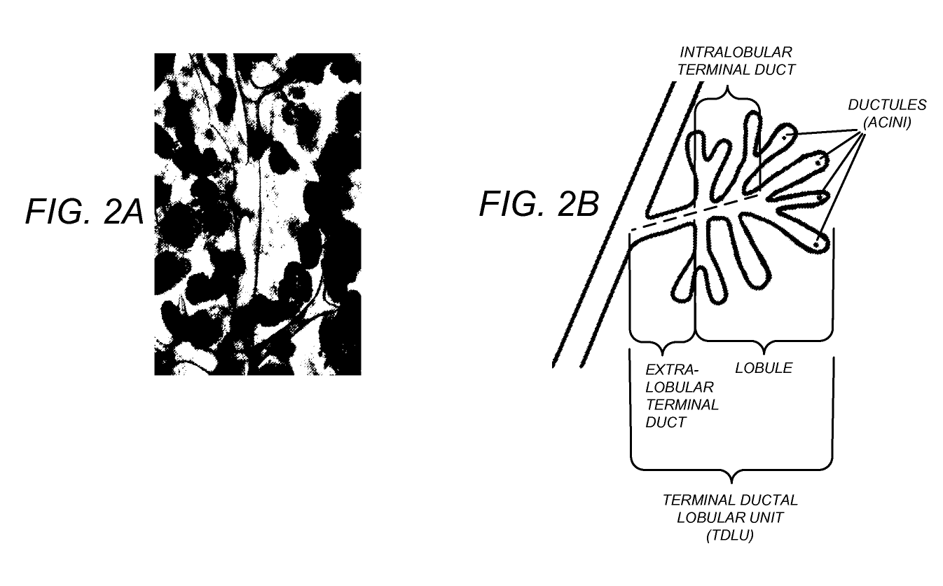

FIGS. 2A-2B illustrate information about TDLU structures of the breast as described in Tabar, et. al., Breast Cancer: The Art and Science of Early Detection with Mammography, Georg Thieme Verlag (2005) (hereinafter “Tabar 2005”). FIG. 2A illustrates an actual TDLU from biopsied tissue, while FIG. 2B illustrates a conceptual drawing of a TDLU. The mature human female breast contains thousands of hormone-sensitive, potentially milk-producing micro-organs called lobules. A terminal duct attached to the main duct system drains each lobule. It is called the terminal ductal lobular unit (TDLU), which normally regresses at menopause. Most breast diseases except papillomas in major ducts arise in terminal ductal lobular units.

The size and shape of the individual lobules vary, but most lobules are between 0.5 and 1.0 mm in diameter. The numerous acini within each lobule can be discerned and the associated terminal duct is easily visualized using subgross, thick-section histology. According to Tabar 2005, the average size of the individual lobule and the mean number of acini within individual lobules in the breast of an adult, premenopausal, nonpregnant woman will change during the phases of the menstrual cycle, with examples of average sizes being 1.06 mm in the proliferative phase, 1.24 mm in the early secretory phase, 1.82 mm in the late secretory phase, and 1.29 mm in the premenstrual phase. There is a complex coexistence of proliferation and regression of the lobules during the menstrual cycle. Beginning a few days before ovulation in each menstrual cycle, clusters of cells bud from the smaller ducts, form lumens, and grow rapidly into fully developed lobules and clusters. If pregnancy does not supervene, the newly formed structures atrophy over several months. Throughout menstrual life, there is almost continuous production and loss of tissue. A single biopsy of the breast may show budding lobules and mature lobules in various stages of regression. In the event of pregnancy, lobule formation and proliferation is intensified and becomes widespread throughout the breast

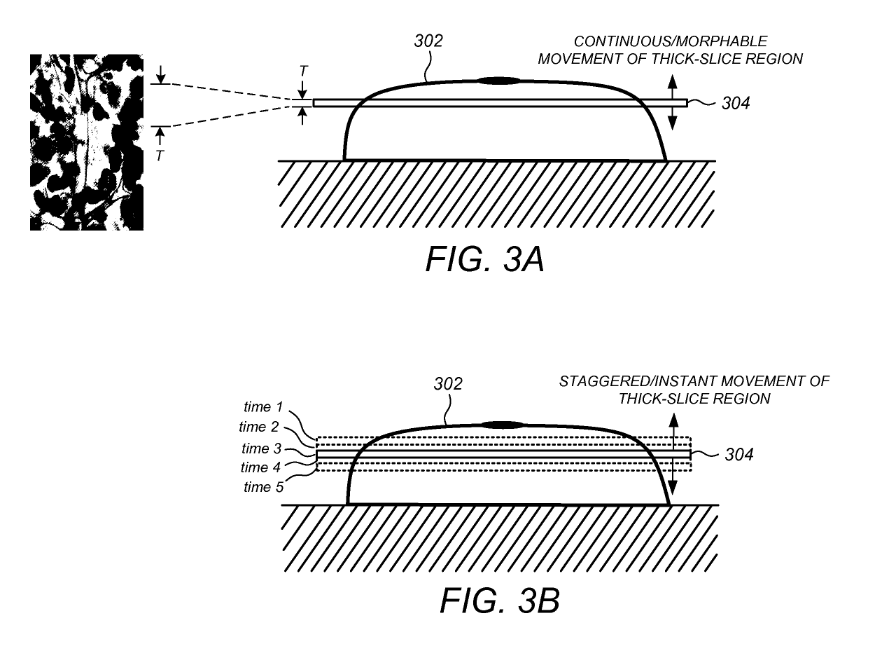

FIG. 3A illustrates a conceptual example of a side view of a thick-slice subvolume 304 of a breast 302, having a thickness T of about 2 mm. In other preferred embodiments, the thickness T can be a relatively small multiple of an average TDLU size which is often in a range of 0.5 mm-1.0 mm or 0.7 mm-1.2 mm, the relatively small multiple (such as 2 or 3) tending to result in the best visibility of the TDLUs in the thick-slice image. For the preferred embodiment of FIG. 3A, the cine display 124 of the thick-slice image is configured to have a continuous, morphable look and feel, as if thick-slice subvolume 304 is slowly moving in an “analog” manner upward or downward. In one preferred embodiment, the progression of the cine display, i.e., the movement of the thick-slice subvolume 304 toward and/or away from the chest wall, is controlled by the clinician using a trackball, mouse, keyboard, or other input. In another preferred embodiment, the cine display automatically progresses at a predetermined rate, which can be adjusted by the clinician from a default value.

FIG. 3B illustrates a conceptual example of a side view of the thick-slice subvolume 304 corresponding to a staggered or instantaneously-switching cine-like display 124 according to another preferred embodiment. For this case, a first thick-slice subvolume is displayed for certain period, and then the display is instantly switched to the next adjacent thick-slice subvolume in the breast. The turn of a mouse wheel, or click on the keyboard, or turn of a trackball can trigger the instant movement to the next thick-slice image. In other preferred embodiments, the successive thick-slice subvolumes can partially overlap, for assisting in display of features straddling the vertical borders between thick-slice subvolumes. It has been found that a display time of about 2-3 seconds per thick-slice image (e.g., 2-3 seconds per 2-mm thick-slice subvolume) provides for good perception of the breast tissue patterns. For the morphable preferred embodiment of FIG. 3A, this translates to a vertical “speed” of about 1-1.5 seconds per millimeter for the thick-slice subvolume 304.

As described in 2005/0171430A1, supra, a coronal thick-slice image comprises an integration of a plurality of individual ultrasound slices lying within a coronal slab-like subvolume, i.e., coronal thick-slice subvolume. Thus, for example, where a coronal slab-like subvolume is represented by a three-dimensional voxel array V(x,y,z) of scalar values, the corresponding coronal thick-slice image would be a two-dimensional pixel array P_{COR}(x,y) of scalar values. In one preferred embodiment, each pixel value P_{COR}(x,y) is simply computed as an arithmetic average along the corresponding voxel column at (x,y) having the voxel values V(x,y,z₀), V(x,y,z₁), V(x,y,z₂), . . . , V(x,y,z_{N}), where N is the number of individual ultrasound slices lying in the coronal slab-like subvolume. Other techniques for integrating the component ultrasound slices into the coronal thick-slice images P_{COR}(x,y) according to the preferred embodiments include arithmetic averaging, geometric averaging, reciprocal averaging, exponential averaging, and other averaging methods, in each case including both weighted and unweighted averaging techniques. Other suitable integration methods may be based on statistical properties of the population of component ultrasound slices at common locations, such as maximum value, minimum value, mean, variance, or other statistical algorithms.

There can be a combination of both art and science in the perception of tissue structures within the breast by the clinician. The preferred embodiments described herein have been found to be strikingly advantageous for clinician perception of patterns relevant to the detection of breast abnormalities, such as those caused by cancer, by cueing to the human's ability to perceive certain types of visual harmonies and, more importantly, disturbance of those harmonies, even among otherwise random arrangements. A yard full of flowers, for example, might have a random arrangement of flowers, but the gardener can often detect a disharmony indicating that an irrigation pipe is leaking, or a disharmony indicating that an animal has tromped through, and so forth. In a similar way, cueing the thicknesses of the ultrasonic thick-slice subvolumes to observe TDLU patterns and structures provides for an analogous detection of disharmony there among by clinicians familiarized with normal, harmonious patterns and structures. The use of coronal thick-slice images is particularly useful because the ducts of the breast, to which the TDLUs are connected, all lead to the nipple, so there is an expected progression of their locations in the coronal thick-slice images to assist in the detection of the harmonies/disharmonies among the TDLUs, linear densities, and connective tissues.

As described previously, it is preferable that the breast have been chestwardly compressed during the acquisition of the volumetric ultrasound scans. One particular advantage of such chestward compression is that spiculated lesions will tend to flatten out in a direction generally parallel to the coronal plane. This, in turn, causes the spiculation structures to be more readily visible in the coronal thick-slice images.

The preferred embodiments described thus far in the instant specification have dealt primarily with coronal thick-slice imaging for a chestwardly-compressed breast. According to another preferred embodiment, it has also been found useful to display thick-slice images corresponding to slab-like subvolumes parallel to other planes, such as the craniocaudal (CC) and/or mediolateral oblique (MLO) planes (which are standard to x-ray mammography), even though the breast volume was not flattened along those planes and was instead compressed in the chestward direction when the ultrasound volume was acquired. Such thick-slice images are referenced herein as CC-projection thick-slice images, MLO-projection thick-slice images, LAT-projection thick-slice images, and so on, the term “projection” in these instances being used to signify that the breast was compressed along a plane that is not “native” to that view, i.e., a plane that is not parallel to the plane against which the breast is traditionally compressed for that view. It has been found that, especially when viewed in conjunction with the coronal thick-slice images, the CC-projection thick-slice images, MLO-projection thick-slice images, and/or other projection thick-slice images further facilitate enhanced visualization of one or more breast cancer subtypes. As with the coronal thick-slice images, it has been found particularly advantageous for the thicknesses of the corresponding slab-like subvolumes to be related to TDLU size in a manner similar to the thicknesses of the coronal slab-like subvolumes, supra.

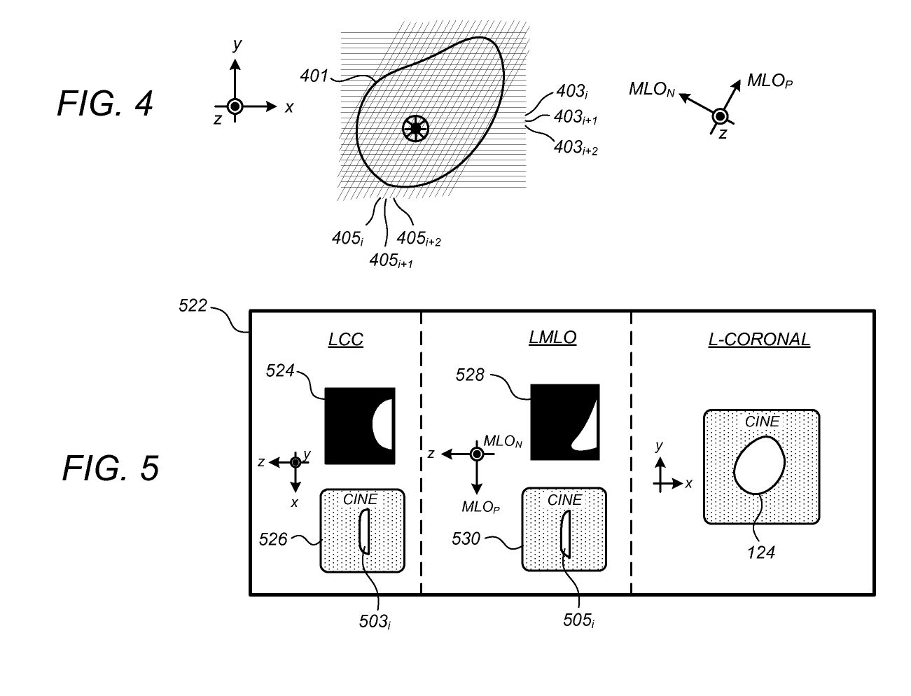

FIG. 4 illustrates a conceptual front view of a breast ultrasound volume 401 acquired by ultrasonic scans of a chestwardly-compressed breast. For simplicity, the description herein refers only to a left breast volume, it being understood that analogous processing and displaying is performed for the right breast. According to a preferred embodiment, for CC-projection thick-slice images, the breast ultrasound volume 401 is considered as being divided into a plurality of CC-projection thick-slice regions 403₁. For an exemplary 2 mm slab thickness, there would be about 75 thick-slice regions 403₁, 403₂, . . . 403₇₅ if the compressed breast extends 15 cm in the vertical (head-to-toe) direction. Similarly, for MLO-projection thick-slice images, the breast ultrasound volume 401 is considered as being divided into a plurality of MLO thick-slice regions 405ᵢ as shown in FIG. 4. It is to be appreciated that, as stated previously, the breast is not compressed along CC or MLO planes for the CC-projection thick-slice images and the MLO-projection thick-slice images, respectively, but rather is compressed in a chestward direction.

FIG. 5 illustrates a user display 522 according to a preferred embodiment, comprising different areas for viewing CC information and MLO information in addition to the coronal thick-slice image cine display 124 described supra. In the CC information area, a CC-view x-ray mammogram 524 is provided, either digitally on a monitor or using film placed on a light box. Positioned near the CC-view x-ray mammogram 524 is a CC-projection thick-slice display 526, providing a cine-like display of CC-projection thick-slice images 503₁ corresponding respectively to the CC-projection thick-slice regions 403₁. The CC-projection thick-slice images 503ᵢ are preferably displayed with a similar look-and-feel as the coronal thick-slice image display 124. Optionally, a nearby additional display (not shown) comprising a side-by-side array of a plurality of the CC-projection thick-slice images 503₁ can be provided.

Further improved perception of the breast structures and anatomical abnormalities therein is provided by the proximal display of the CC-projection thick-slice images 503₁ and the CC-view x-ray mammogram 524. Advantageously, the single volumetric ultrasound scan 401 used for the coronal thick-slice images also provides the basis for the CC-projection thick-slice images 503ᵢ, such that additional volumetric ultrasound scanning along additional planes of compression is not required. Although it is preferable that the breast ultrasound volume 401 and the CC-view x-ray mammogram 524 were acquired in a same clinical visit, the scope of the preferred embodiments is not so limited, and indeed a variety of temporal combinations of the displayed information is within the scope of the preferred embodiments. Also illustrated in FIG. 5 is an analogous MLO information area in which an MLO-view x-ray mammogram 528 is provided near an MLO-projection thick-slice display 530, providing a cine-like display of MLO-projection thick-slice images 505₁ corresponding respectively to the MLO-projection thick-slice regions 405₁.

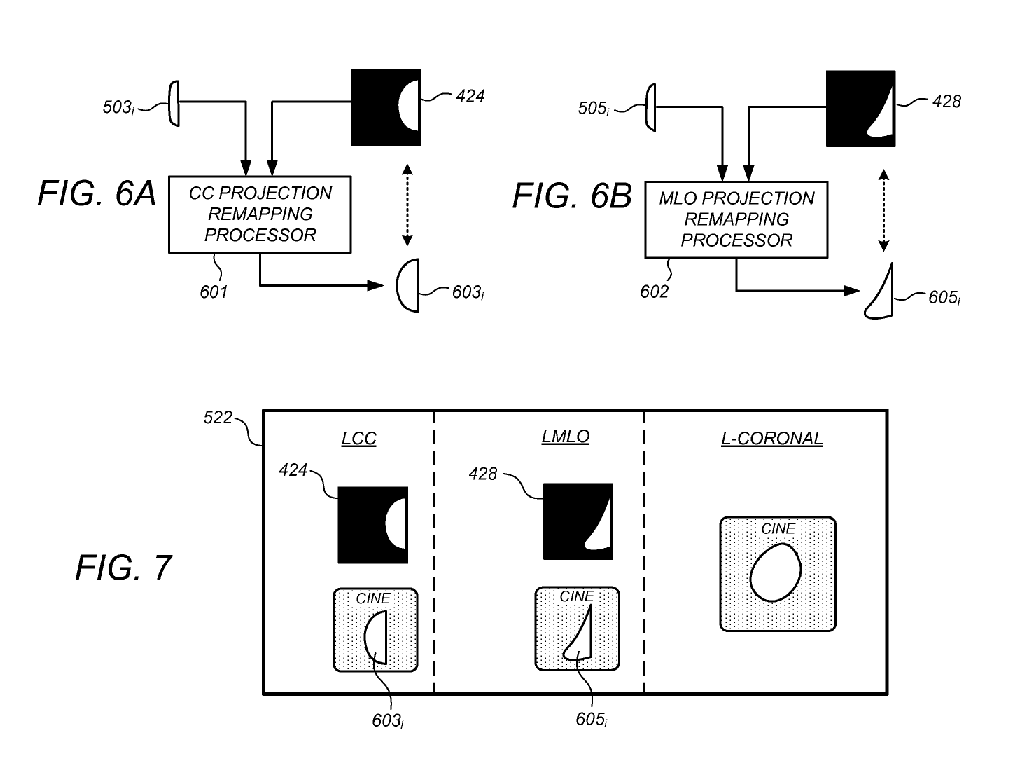

FIG. 6A conceptually illustrates additional improved breast ultrasound processing according to a preferred embodiment, wherein the CC-projection thick-slice images 503₁ are remapped (e.g., by stretching/morphing) by a CC-projection remapping processor 601 to result in remapped CC-projection thick-slice images 603₁ that more closely correspond to the CC-view x-ray mammogram 424 in shape. Because the CC-projection thick-slice regions 403₁ are taken from a chestwardly-compressed breast, the resultant CC-projection thick-slice images 503₁ (see FIG. 5, supra) are necessarily compacted or “smooshed” toward the chest wall, which makes their profile look different from what a more conventional CC-view x-ray mammogram looks like. The remapped images 603₁ are more easily compared to the CC-view x-ray mammogram 424 than the non-remapped (“smooshed”) images 503 further facilitating perception and analysis of the breast tissue. Additionally, time and effort are saved in the ultrasound-to-x-ray comparison process, and the overall user interface is more desirable for radiologists and hospital equipment-purchasing personnel. FIG. 6B illustrates analogous processing of the MLO-projection thick-slice images 505 which are remapped by an MLO-projection remapping processor 602 to result in remapped MLO-projection thick-slice images 605₁ that more closely correspond to the MLO-view x-ray mammogram 428.

FIG. 7 illustrates the user display 522 with the remapped CC-projection thick-slice images 603₁ and the remapped MLO-projection thick-slice images 605₁ displayed next to the CC-view x-ray mammogram 424 and the MLO-view x-ray mammogram 428, respectively, the usefulness of the remapping process being intuitively evident when contrasted with the non-remapped versions of FIG. 5, supra. The remapping algorithms can be selected from any of a variety of known algorithms capable of geometrically registering different images of the same body part. Optionally, the remapping algorithm for any particular thick-slice image 503ᵢ or 505₁ can be neighborhood based, i.e., the remapping parameters can also depend from adjacent thick-slice regions in the breast or from the entire breast volume.

Preferably, the x-ray mammogram views (or digitized versions thereof if in film format) are provided as inputs to the remapping algorithms to facilitate the remapping, as illustrated in FIGS. 6A and 6B. In one example, the nipple and skinline for each of the x-ray mammograms and the unmapped thick-slice images are segmented using known methods, and the remapping is based upon stretching the thick-slice image skinline and nipple out to the x-ray mammogram skinline and nipple, respectively. In an alternative preferred embodiment, the x-ray mammograms are not used as inputs to the remapping process, and other information (for example, the pounds of chestward compression applied in conjunction with a deformable model of the breast) or simple thumbnail estimates, either fixed or user-adjustable, are used for the remapping process.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, although primarily described supra in the context of ultrasound imaging, it is to be appreciated that data from other full-field breast imaging modalities (e.g., MRI, CT, PET) can be advantageously processed and displayed according to one or more of the described preferred embodiments. By way of further example, it is to be appreciated that substantially parallel to a coronal plane is used herein to generally reflect the practical realities of situations such as head-on scanning of the breast, and that there may be some deviation from the plane of the chest wall. For example, for a particular patient having highly pendulous breasts it might be found most optimal to compress the breast at some small angle, such as 15 degrees, away from the plane of the chest wall. In this case, slab-like subvolumes that are taken parallel to the plane of compression would still be considered substantially parallel to the coronal plane.

By way of still further example, although it is particularly advantageous to incorporate chestward breast compression and coronal thick-slice viewing of the TDLU-size-related thick-slice images, it is to be appreciated that the scope of the preferred embodiments is not necessarily so limited. In other preferred embodiments, for example, the breast could be compressed along an MLO or CC plane and the thick-slice subvolumes parallel to the MLO or CC plane, respectively. In still other preferred embodiments, compression could be omitted.

By way of even further example, it is to be appreciated that the scope of the preferred embodiments is not necessarily limited to choosing thick-slice thicknesses that optimize TDLU viewing, but rather can include any type of selection and processing of the thick-slice subvolume data that results in thick-slice images from which TDLU patterns are emphasized or otherwise readily apparent. For example, in alternative preferred embodiments, the thick-slice subvolumes can be thicker, but are processed (e.g., using CAD or other intelligent processing or voxel weighting scheme) such that TDLUs are emphasized. In one example, a structural emphasis algorithm similar to that described in the commonly assigned U.S. Pat. No. 7,103,205, which is incorporated by reference herein, can be adapted for emphasizing the TDLU structures. Therefore, reference to the details of the preferred embodiments are not intended to limit their scope, which is limited only by the scope of the claims set forth below.

What is claimed is:

1. A method for processing breast ultrasound information, comprising:
receiving a three-dimensional data volume of a sonographic property of a chestwardly compressed breast;
processing the data volume to generate a plurality of two-dimensional coronal thick-slice images, each coronal thick-slice image being representative of the sonographic property within a thick-slice subvolume of the breast substantially parallel to a coronal plane; and
displaying the plurality of coronal thick-slice images to a viewer;
wherein each of said thick-slice subvolumes has a thickness such that terminal ductal lobular units (TDLUs) within the breast are readily apparent in said coronal thick-slice images.

2. The method of claim 1, wherein said thickness is roughly 2 mm.

3. The method of claim 2, wherein said thick-slice images are displayed in a cine progression.

4. The method of claim 3, wherein said cine progression is selected from the group consisting of staggered progression and morphable continuous progression.

5. A method for processing breast ultrasound information, comprising:
receiving a three-dimensional data volume of a sonographic property of a breast;
processing the data volume to generate a plurality of two-dimensional coronal thick-slice images, each coronal thick-slice image being representative of the sonographic property within a thick-slice subvolume of the breast substantially parallel to a coronal plane; and
displaying the plurality of coronal thick-slice images to a viewer;
wherein each of said thick-slice images is computed to emphasize terminal ductal lobular units (TDLUs) at least partially contained in the corresponding thick-slice subvolume.

6. The method of claim 5, wherein said sonographic property of the breast is derived from ultrasonic scans of the breast while chestwardly compressed.

7. The method of claim 6, wherein each of said thick-slice subvolumes is roughly 2 mm in thickness.

8. The method of claim 7, wherein said thick-slice images are displayed in a cine progression.

9. The method of claim 8, wherein said cine progression is selected from the group consisting of staggered progression and morphable continuous progression.

10. A computer program product embodied in an information storage device containing instructions to a breast ultrasound information processing and display system to:
receive a three-dimensional data volume of a sonographic property of a chestwardly compressed breast;
process the data volume to generate a plurality of two-dimensional coronal thick-slice images, each coronal thick-slice image being representative of the sonographic property within a thick-slice subvolume of the breast substantially parallel to a coronal plane; and
display the plurality of coronal thick-slice images to a viewer;
wherein each of said thick-slice subvolumes has a thickness of about 2 mm;
and wherein said thick-slice images are displayed in a cine progression selected from the group consisting of a staggered cine progression and a morphable continuous cine progression.

11. A breast ultrasound system, comprising:
an ultrasound acquisition unit compressing a breast in a chestward direction and ultrasonically scanning the breast to generate a three-dimensional data volume of a sonographic property of the chestwardly compressed breast;
a processing unit processing the data volume to generate a plurality of two-dimensional projection-view thick-slice images, each projection-view thick-slice image being representative of the sonographic property within a thick-slice subvolume of the breast substantially parallel to a projection-view plane substantially different than a coronal plane; and
a display unit displaying the plurality of projection-view thick-slice images to a viewer;
wherein each of said thick-slice subvolumes has a thickness such that terminal ductal lobular units (TDLUs) within the breast are readily apparent in said coronal thick-slice images.

12. The breast ultrasound system of claim 11, wherein said thickness is roughly 2 mm.

13. The breast ultrasound system of claim 12, wherein said ultrasound acquisition unit comprises a scanning pod, the scanning pod comprising:
a substantially taut membrane, a bottom surface of the membrane contacting the breast to compress the breast in said chestward direction; and
a linear ultrasound probe that is automatically translated across a top surface of the membrane to acquire scans of the breast therethrough.

14. The breast ultrasound system of claim 12, wherein said projection-view plane comprises a standard mammographic view plane selected from the group consisting of CC, MLO, and LAT, and wherein said display unit displays said projection-view thick-slice images sufficiently close to a corresponding-view x-ray mammogram to allow simultaneous viewing thereof.

15. The breast ultrasound system of claim 14, further comprising a projection remapping processor remappably processing each of said plurality of projection-view thick-slice images to have a lateral shape that more closely corresponds to the corresponding-view x-ray mammogram for facilitating comparison to the corresponding-view x-ray mammogram.

16. The breast ultrasound system of claim 14, wherein said display unit displays said projection-view thick-slice images in one of a staggered cine progression and a morphable continuous cine progression.

17. A method for projection-view processing and display of breast ultrasound information, comprising:
receiving a three-dimensional data volume of a sonographic property of a chestwardly compressed breast;
processing the data volume to generate a plurality of two-dimensional projection-view thick-slice images, each projection-view thick-slice image being representative of the sonographic property within a thick-slice subvolume of the breast substantially parallel to a projection-view plane substantially different than a coronal plane; and
displaying the plurality of projection-view thick-slice images to a viewer;
wherein each of said thick-slice subvolumes has a thickness such that terminal ductal lobular units (TDLUs) within the breast are readily apparent in said coronal thick-slice images.

18. The method of claim 17, wherein said thickness is roughly 2 mm.

19. The method of claim 17, wherein said projection-view plane comprises a standard mammographic view plane selected from the group consisting of CC, MLO, and LAT, and wherein said projection-view thick-slice images are displayed sufficiently close to a corresponding-view x-ray mammogram to allow simultaneous viewing thereof.

20. The method of claim 19, further comprising remappably processing each of said plurality of projection-view thick-slice images to have a lateral shape that more closely corresponds to the corresponding-view x-ray mammogram, the resultant remapped projection-view thick-slice image thereby being more easily compared to the corresponding-view x-ray mammogram.
